(19) United States
(12) Patent Application Publication
(10) Pub. No.: US 2008/0130266 A1
(43) Pub. Date: Jun. 5, 2008

(54) Fragrancer

(75) Inventors: Donald E. DeWitt, Syracuse, IN (US); Daniel A. Heuer, New Carlisle, IN (US); Joshua E. DeWitt, Syracuse, IN (US)

Correspondence Address: CARY R. REEVES, 13902 Meadowbrook Drive, Broomfield, CO, 80020, US

(73) Assignee: INNOVATIVE INSTRUMENTS, INC., Syracuse, IN (US)

(21) Appl. No.: 11/946,047

(22) Filed: Nov. 28, 2007

Related U.S. Application Data
(60) Provisional application No. 60/872,858, filed on Dec. 5, 2006.

(51) Int. Cl. A61L 9/03; A61L 9/015; F21V 33/00

(52) U.S. Cl. 362/96; 422/123; 422/125

(57) ABSTRACT

The present invention provides a fragrancer for dispersing fragrance into the air. In one aspect of the invention, the fragrancer includes a base and a modular scent holder interchangeably mounted to the base. In another aspect of the invention, the base includes a light producing element. In another aspect of the invention, the fragrancer includes a power source and a powered device and a modular scent holder and base define an interlock operable to enable power to the powered device when the modular scent holder is seated on the base and operable to interrupt power to the powered device when the modular scent holder is unseated from the base. In another aspect of the invention, the fragrancer includes a decorative element such as a globe, globe insert, or trim ring interchangeably mounted to the base. In another aspect of the invention, a modular scent holder interchangeably mountable to a fragrancer includes a first wall defining an opening able to receive a portion of the fragrancer and the first wall is able to support a volatile fragrance producing substance.

### FIELD OF THE INVENTION

The invention relates to fragrancers for dispersing fragrance into the air.

### BACKGROUND

Fragrancers have been produced in a variety of configurations. Fragrancers generally include a reservoir containing a volatile fragrance and a wick extending from the reservoir. Dispersion of the fragrance is often aided by a heater or fan to increase the rate of evaporation of the fragrance.

### SUMMARY OF THE INVENTION

The present invention provides a fragrancer for dispersing fragrance into the air.

In one aspect of the invention, the fragrancer includes a base and a modular scent holder interchangeably mounted to the base. The scent holder includes a base wall defining an opening able to receive a portion of the base, and a volatile fragrance producing substance supported by the modular scent holder.

In another aspect of the invention, the fragrancer includes an inner side wall extending away from the base wall and an outer side wall extending away from the base wall. The base wall, inner side wall, and outer side wall define an annular tray surrounding the opening able to contain the volatile fragrance producing substance.

In another aspect of the invention, the base includes a light producing element and the scent holder is receivable by the base with the light producing element extending through the opening in the base wall.

In another aspect of the invention, the fragrancer includes a power source and a powered device. The modular scent holder and base define an interlock operable to enable power to the powered device when the modular scent holder is seated on the base and operable to interrupt power to the powered device when the modular scent holder is unseated from the base.

In another aspect of the invention, the fragrancer includes a decorative element such as a globe, globe insert, or trim ring interchangeably mounted to the base. The decorative element is provided in a variety of decorative appearances and is interchangeable by a consumer to vary the decorative appearance of the fragrancer.

In another aspect of the invention, the fragrancer includes a globe, a power source, and a powered device. The globe and base define a switch operable to enable power to the powered device when the globe is in a first position and interrupt power to the powered device when the globe is in a second position.

In another aspect of the invention, the fragrancer includes a globe mounted to the base for rotation relative to the base. The fragrancer is responsive to rotation of the globe on the base to change its fragrance output.

In another aspect of the invention, a modular scent holder interchangeably mountable to a fragrancer includes a first wall defining an opening able to receive a portion of the fragrancer and the first wall is able to support a volatile fragrance producing substance.

### BRIEF DESCRIPTION OF THE DRAWINGS

Various examples of the present invention will be discussed with reference to the appended drawings. These drawings depict only illustrative examples of the invention and are not to be considered limiting of its scope.

FIG. 1 is a perspective view of a fragrancer according to the present invention;

FIG. 2 is an exploded perspective view of the fragrancer of FIG. 1;

FIG. 3 is a side elevation view of the fragrancer of FIG. 1;

FIG. 4 is a cross-sectional view taken along line 4-4 of FIG. 3;

FIG. 5 is a cross-sectional view taken along line 5-5 of FIG. 3 showing a globe rotated to a first position;

FIG. 6 is a cross-sectional view as in FIG. 5 showing a globe rotated to a second position;

FIG. 7 is a cross-sectional view of a scent pack component of FIG. 2;

FIG. 8 is a cross-sectional view of a scent pack component of FIG. 2 illustrating an alternative wall configuration;

FIG. 9 is a cross-sectional view similar to that of FIG. 4 with the scent pack of FIG. 8;

FIG. 10 is a circuit diagram of a flame simulation circuit;

FIG. 11 is a circuit diagram of a flame simulation circuit;

FIG. 12 is a process flow diagram of a flame simulation produced by the flame simulation circuit of FIG. 11; and

FIG. 13 is a chart depicting a sine² curve illustrating one aspect of the flame simulation circuit of FIG. 11.

### DESCRIPTION OF THE ILLUSTRATIVE EXAMPLES

Embodiments of the fragrancer according to the present invention include a fragrance generator. The fragrance generator may use a volatile gel, a volatile oil, and/or other fragrance producing substances (fragrance). The fragrance generator may include a heating element, a fan, a wick, and/or other suitable device to vaporize the fragrance and emit it into the surrounding air. The fragrance may be provided in a modular scent pack that is easily changed to replenish the fragrance and/or allow changing the scent. The scent pack may include a central opening for receiving a light source to simulate, e.g., a candle flame at the center of a wax candle. The central opening may be round, rectangular, or any other shape. The central opening may include a projection that extends upwardly around the light source to provide a light diffuser, a color filter, a handle, a protective shield to prevent volatile fragrances from soiling the light source, and/or to provide other functions. The projection may be open ended or it may be closed. For example, a scent pack may be provided that includes an annular tray open at one end and including a flame shaped central projection that defines a socket for receiving a light source.

The fragrancer may advantageously include a modular construction to permit customization during manufacturing to address different markets and/or seasonal needs. The modular parts may also be provided to the end user to allow the end user to customize the fragrancer. Modular components may include globes, globe inserts, trim rings, scent packs, and/or other modular components. For example, globes with varying colors, patterns, shapes, and/or textures may be provided. For example, seasonal globes with printed or painted holiday scenes may be provided to allow the fragrancer to be adapted for the season. A globe is preferably translucent or transparent and allows some light to pass through it. The globe may be made of glass, plastic, paper, wood, ceramic, or any other suitable material. For materials that are generally more opaque, such as wood, the globe may have a thin cross section so that the material is translucent. Similarly, inserts may be provided that insert between the globe and light source. The inserts may include diffusers, color filters, light passages for projecting light images onto the globe, opaque patterns for projecting shadows onto the globe, and/or other inserts. The inserts may be made of glass, plastic, paper, wood, ceramic, or any other suitable material. The fragrancer may incorporate a modular trim ring that covers a base and is changeable to vary the color, shape, decorative patterns, and/or other aspects of the base. The trim ring may be made of glass, plastic, paper, wood, ceramic, or any other suitable material.

The fragrancer may be activated in a variety of ways. For example, it may be always on whenever a power source is connected to it. Alternatively, it may have an on/off control. The on/off control may be activated by one or more elements of the fragrancer assembly such that the fragrancer will only operate when properly assembled. For example the on/off control may form an interlock with one or more elements such as the scent pack and/or globe. Thus, if the fragrancer is tipped over or otherwise disturbed causing the interlocked element to be displaced the fragrancer will shut off. Such an interlock may include switches activated by depressing a button, magnetic forces, photo sensors, and/or other suitable switches. For example, one or more magnetically sensitive switches can be incorporated into the fragrancer circuitry such that a magnet attached to the globe and/or scent pack must be properly positioned for the fragrancer to operate. A switch may also be incorporated such that rotation of an element of the fragrancer activates the fragrancer. For example, a magnet attached to the globe may activate the fragrancer when the globe is rotated to a predetermined position.

The fragrancer may also include a mechanism to modulate the fragrance output from the scent pack. The mechanism may include varying the temperature of the scent pack, varying the airflow around the scent pack, and/or other mechanisms. For example, the scent pack may be heated by an adjustable heat source. The airflow around the scent pack may be varied by changing the speed of a fan. The airflow around the scent pack may be varied by changing the size of air intake openings in the fragrancer. Any one of these mechanisms may be used alone or they may be used in any combination. For example, the temperature of the heat pack and the size of air intake openings at the base of the fragrancer may be varied together to modulate the fragrance. For example, rotationally adjustable air intake openings may be incorporated into the globe and the fragrancer base and a scent pack heater may be indirectly modulated by a Hall Effect device such that rotating the globe modulates the heater and varies the air intake openings. In another example, the circuit may keep track of the time that the fragrancer is activated and incrementally increase heat to compensate for diminishing fragrance output from the scent pack.

The illustrative fragrancer includes an optional light source and is described as a flameless candle. While this is one preferred embodiment of the invention, the fragrancer may be provided without the light source.

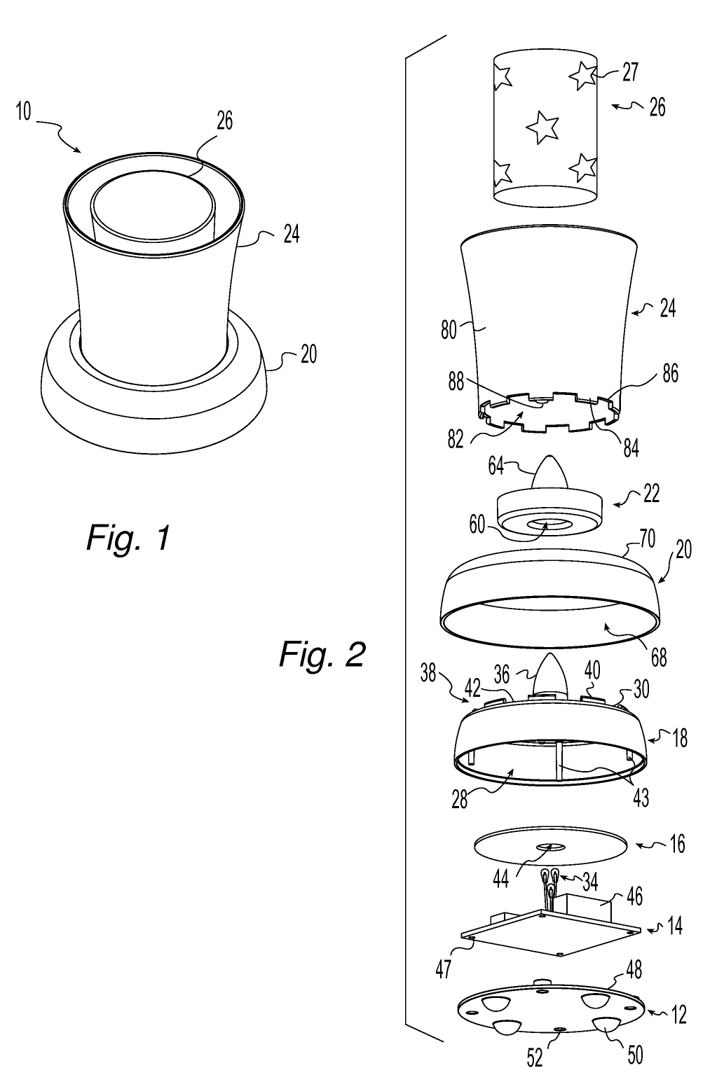

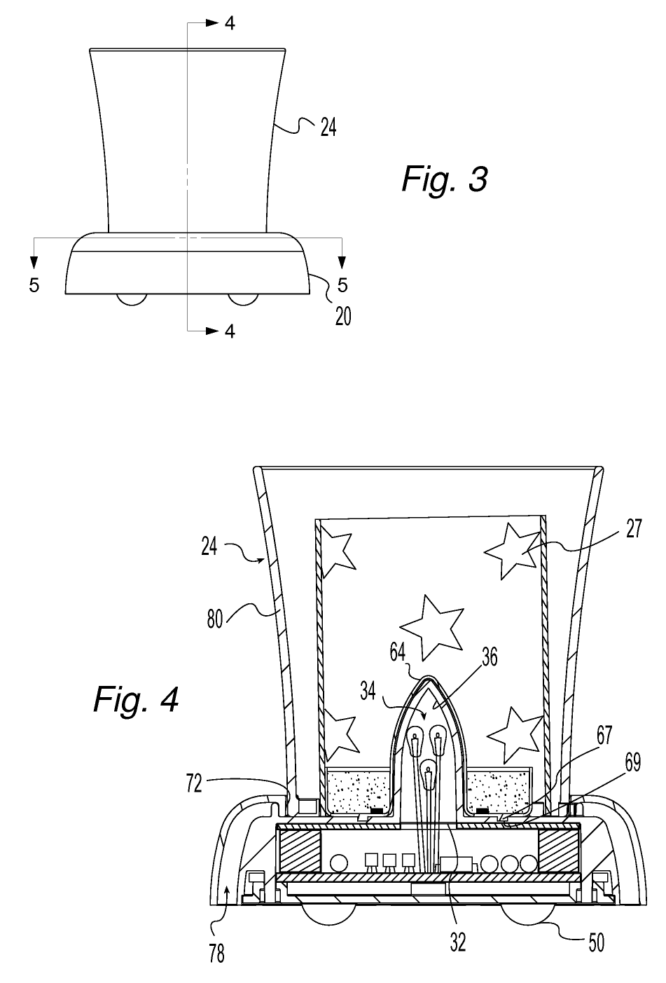

The illustrative fragrancer, in this example a flameless candle assembly 10, of FIGS. 1-7, includes a base plate 12, a circuit board 14, a heat plate 16, a luminary base 18, a trim ring 20, a scent pack 22, a globe 24, and a globe insert 26. The base plate, 12, circuit board 14, and heat plate 16, are mounted to the luminary base 18 to form a base assembly. The trim ring 20, scent pack 22, globe 24, and globe insert 26 rest loosely over the luminary base 18 and are easily exchanged to create different effects.

The luminary base 18 defines a hollow shell having an open bottom 28 and a closed top 30. The top 30 includes a central opening 32 (FIG. 4) for receiving an array of bulbs 34. In the illustrative embodiment, a bulb shroud 36 surrounds the central opening 32 and extends upwardly generally in the shape of a candle flame. The bulb shroud 36 is translucent and diffuses the light from the array of bulbs 34. The bulb shroud 36 may optionally be tinted to act as a color filter for changing the color of the simulated flame. An interrupted ring 38 projects upwardly from the top 30 of the luminary base 18. The ring 38 includes tabs 40 and intervening notches 42 and forms part of an adjustable air passage through the candle 10. Stakes 43 project inside the luminary base 18 and terminate near the bottom 28. Preferably, the luminary base 18 is molded from plastic.

The heat plate 16 is a circular disc having a central opening 44 for receiving the array of bulbs 34. The heat plate 16 is preferably made of a conductive material so that it readily transmits heat. In the illustrative example, the heat plate 16 is made of aluminum.

The circuit board 14 includes a heating element and a flame simulation circuit. In the illustrative example, the heating element includes a pair of power resistors 46. The flame simulation circuit drives the array of bulbs 34 as will be explained further below. The circuit board includes holes 47 configured to align with the stakes 43 of the luminary base 18.

The base plate 12 includes a generally planer disc 48, depending molded feet 50, and holes 52 configured to align with the stakes 43 of the luminary base 18.

The base is assembled by placing the heat plate 16 through the bottom 28 of the luminary base 18 and into contact with the underside of the top 30. The circuit board 14 is next placed through the bottom 28 of the luminary base 18. The bulbs 34 are inserted through the heat plate 16 and into the bulb shroud 36. The holes 47 engage the stakes 43. The base plate 12 is then placed through the bottom 28 of the luminary base 18 with the holes 52 engaged with the stakes 43. The base plate 12 is slid over the stakes 43 to abut the circuit board 14 and press the power resistors 46 firmly against the heat plate 16. The stakes 43 are then heat deformed to hold the assembly together. In this way, a tight fit of the resistors 46 against the heat plate 16 is assured.

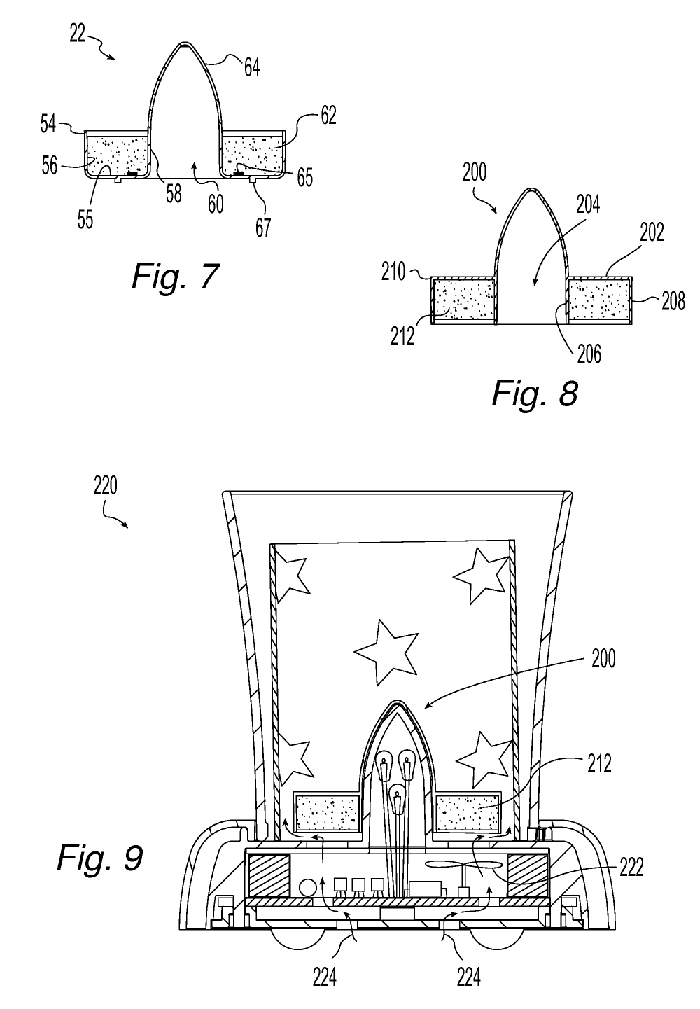

The scent pack 22 (see FIG. 7) includes a base wall 55 defining an opening 60. An inner side wall 58 extends away from the base wall 55 and surrounds the opening 60. An outer side wall 56 extends away from the base wall 55 and surrounds the inner side wall 58 such that the base wall 55, inner side wall 58, and outer side wall 56 define an annular tray 54 surrounding the opening 60. In the embodiment of FIG. 7, the base wall 55 is in the form of a bottom wall and the side walls 56, 58 extend upwardly to define an upwardly opening annular tray 54. While shown with cylindrical side walls, the scent pack 22 sidewalls may be any shape. For example, the side walls may be triangular, rectangular, hexagonal, or be in the shape of any other polygon, regular curve, irregular curve, and or random shape. The scent pack 22 is filled with a volatile fragrance 62 such as an aromatic gel, oil, wax and/or other suitable fragrance. The scent pack 22 may also include an optional wick (not shown). The central opening 60 receives the bulb shroud 36 of the luminary base 18 to simulate a candle flame at the center of a wax candle. In the illustrative scent pack 22, the inner wall 58 extends upwardly to form a central projection 64. The central projection 64 is closed at the top and generally conforms to the shape of the bulb shroud 36. The central projection 64 may be frosted to further diffuse light from the bulbs 34. The central projection 64 may also be tinted to act as a color filter to change the color of the light. Scent packs 22 may be provided in a variety of configurations of scent, light diffusion, and color filtration to allow customization of the light and scent characteristics of the candle. The central projection 64 provides a protective shield to prevent the volatile fragrance 62 from soiling the bulb shroud. Thus, with each change of the scent pack 22, a clean light path is provided. Finally, the central projection 64 provides a handle to facilitate gripping the scent pack 22 for insertion and removal. The scent pack is preferably molded in plastic.

The scent pack 22 is placed over the bulb shroud 36 with the floor 55 of the scent pack 22 in contact with the top 30 of the luminary base 18. The illustrative scent pack 22 includes an optional ring shaped magnet 65 attached to its floor 55 as part of an interlock system to ensure that the flameless candle only operates when the scent pack 22 is properly in place on the flameless candle. The interlock includes a reed switch or other magnetically sensitive circuit component in the flameless candle circuit that turns the flameless candle off when the scent pack is not in position on the luminary base 18. For example, if the flameless candle is tipped over and the scent pack becomes dislodged, the flameless candle will turn off. The illustrative scent pack 22 also includes a downwardly projecting ring 67 molded onto the floor 55. The ring 67 engages a groove 69 in the top of the luminary base 18. The groove 69 contains a switch (not shown) that is activated by the ring 67 pressing downwardly into the groove. The switch turns the flameless candle off when the scent pack is not in position on the luminary base 18. For example, if the flameless candle is tipped over and the scent pack becomes dislodged, the flameless candle will turn off. Both the magnetic interlock and the projecting ring interlock are optional and can be used independently of one another or in combination. Other interlock geometries may be substituted for these including one or more projecting dimples, splines, and/or other geometries. Other interlock devices may be substituted for these including a photo sensor, Hall Effect device, variable resistor, liquid filled switches, and/or other types of devices.

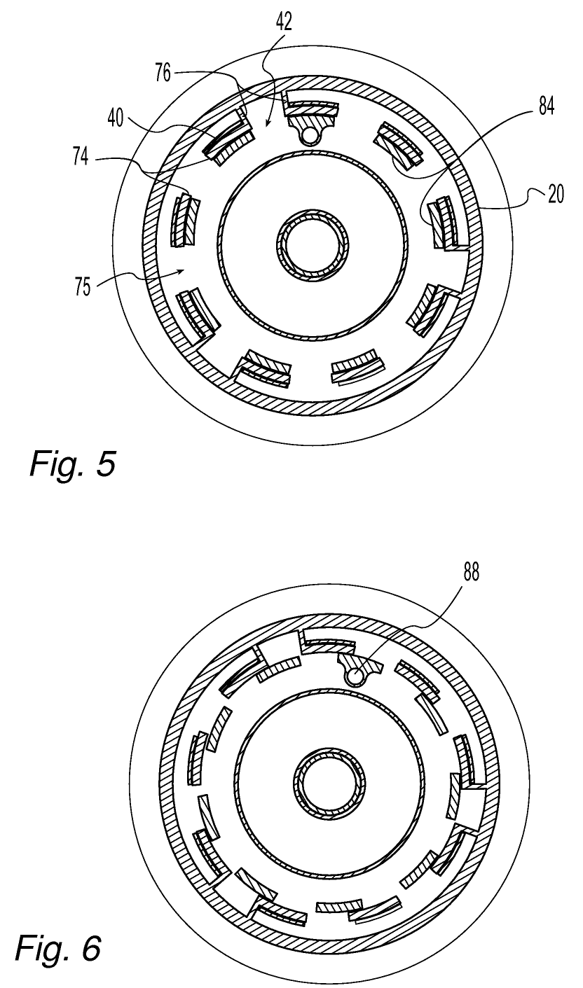

The trim ring 20 defines a hollow shell 66 having an open bottom 68 and a top 70. The top 70 defines a central opening 72 (FIG. 4) sized to receive the globe 24 in sliding fit relationship. The trim ring 20 includes annular tabs 74 (FIG. 5) extending downwardly from the top 70 near the central opening 72 and separated by notches 75. At least some of the tabs 74 connect to radial ribs 76. In the illustrative trim ring 20 of FIG. 5, three pairs of ribs 76 are provided. Each pair of ribs 76 has a spacing less than the width of a corresponding notch 42 between the luminary base tabs 40. The trim ring 20 rests on top of the luminary base 18 with the trim ring tabs 74 providing vertical spacing between the trim ring 20 and the luminary base 18. The ribs 76 provide rotational alignment of the trim ring notches 75 with the luminary base notches 42 to ensure maximum airflow through the aligned notches.

The trim ring 20 and luminary base 18 define an annular air passage 78 (FIG. 4) between them from the bottom of the flameless candle 10 up and around the luminary base and through the notches 42, 75. The feet 50 of the base plate 12, elevate the luminary base 18 and trim ring 20 above the counter surface to provide for air entry into the air passage 78 through the bottom of the flameless candle 10. The trim ring 20 may be provided in a variety of styles, colors, textures, and/or other characteristics to permit customization of the flameless candle 10. The trim ring 20 may include figures, scenes, patterns, and/or other depictions molded into it or applied to it to vary its appearance. For example, various seasonal themes may be printed on the trim ring 20 and used in manufacturing and/or provided to the consumer for seasonal customization. The trim ring is preferably molded from plastic.

The globe 24 includes a generally cylindrical open ended wall 80. The base 82 of the globe 24 defines a ring of alternating tabs 84 and notches 86. The globe 24 rests on top of the luminary base 18 and defines a slip fit inside of the central opening 72 of the trim ring 20. The globe 24 is rotatable relative to the luminary base 18 and trim ring 20 from a first position in which the tabs 84 and notches 86 of the globe 24 align with the tabs and notches of the trim ring 20 and luminary base 18 (FIG. 5) and a second position in which the tabs 84 of the globe 24 align with the notches of the trim ring 20 and luminary base 18. The first position provides relatively more air flow through the air passage 78 and the second position provides relatively less flow through the air passage 78. The globe 24 is continuously adjustable from a fully open air flow position to a fully closed position.

The illustrative globe 24 includes a magnet 88 attached to one of the tabs 84. The flameless candle 10 includes a magnetically sensitive switch responsive to the presence of the magnet 88 to turn the flameless candle on. The switch may be a reed switch, a Hall Effect device, and/or other magnetically sensitive device. The globe is rotatable between an off position in which the magnet 88 is spaced from the switch and an on position in which the magnet 88 is near the switch. Preferably the magnet 88 activates the switch over a rotational range so that the flameless candle 10 is turned on over the range of airflow adjustment depicted in FIGS. 5 and 6. The switch, or another one adjacent to it, may also be responsive to the magnet's 88 position to modulate the scent output of the flameless candle. For example, the flameless candle circuit may include a Hall Effect device, an array of reed switches, and/or other magnetically sensitive circuit component, that varies a circuit parameter in response to the globe's position to vary the amount of heat applied to the scent pack 22 and/or to vary the speed of an optional fan attached to the flameless candle. The circuit response may also be coordinated with the air flow openings as varied by the globe's tabs 84 such that when the tabs are in a more open position like that of FIG. 5, the active scent producing aspects of the circuit are driven to produce more scent and when the tabs are in a more closed position like that of FIG. 6, the active scent producing aspects of the circuit are driven to produce less scent. Other forms of modulation may be substituted for the magnetically sensitive circuit component such as a rotating potentiometer, an optical sensor, and/or other suitable circuit components. The globe 24 may be provided in a variety of styles, colors, textures, and/or other characteristics to permit customization of the flameless candle 10. The globe 24 may include figures, scenes, patterns, and/or other depictions molded into it or applied to it to vary its appearance. For example, various seasonal themes may be printed on the globe 24 and used in manufacturing and/or provided to the consumer for seasonal customization. The globe is preferably molded from plastic.

The illustrative globe insert 26 is generally in the form of a cylindrical sleeve that fits within the globe 24. However, the globe insert 26 may have any shape that fits inside or outside of the globe 24. The globe insert 26 may be provided in a variety of styles, colors, textures, and/or other characteristics to permit customization of the flameless candle 10. The globe insert 26 may include figures, scenes, patterns, and/or other depictions to vary its appearance. For example, various seasonal themes may be formed as cutouts 27 in the globe insert 26 such that a light pattern corresponding to the theme is projected on the globe 24 in the case of a globe insert 26 placed inside of the globe 24 or such that a lighted cutout scene is directly viewable in the case of a globe insert 26 placed outside of the globe. Similarly, depictions may be created as relatively more opaque areas on the globe insert 26 to cast a corresponding shadow on the globe or produce a backlit silhouette. Likewise, transparent colors may be applied to the globe insert to produce colored depictions. Globe inserts 26 may be used in manufacturing and/or provided to the consumer for customization. The globe insert 26 is preferably molded from plastic.

In use, optional trim rings 20, scent packs 22, globes 24, and globe inserts 26 are positioned on the luminary base 18. The flameless candle 10 is turned on, such as by rotating the globe 24, to activate the flame simulation and heat the power resistors 46. The circuitry on the circuit board 14 activates the bulbs 34 to produce light which is transmitted through the bulb shroud 36, scent pack extension 64, globe insert 26, and globe 24. The heat plate 16 conducts heat from the power resistors 46 to create a relatively uniformly heated heat plate 16. Heat from the heat plate 16 is conducted through the top 30 of the luminary base 18 and the floor of the scent pack 22 to warm the fragrance 62 and disperse it into the air. As the air in the globe 24 warms, convective currents are generated in which warmer air rises and is replaced by cooler air drawn through the annular air passage 78 at the base of the flameless candle 10. Rotating the globe 24 rotates the tabs 84 to provide more or less restriction to the flow of makeup air through the annular passage 78 and consequently the airflow out of the flameless candle and thus modulates the intensity of the scent produced by the flameless candle.

FIGS. 8 and 9 illustrate an alternative arrangement for the scent pack and fragrancer. In the embodiment of FIGS. 8 and 9, the scent pack 200 includes a base wall 202 in the form of a top wall and the inner and outer side walls 206, 208 extend downwardly away from the base wall 202 and surround the opening 204 to form a downwardly opening annular tray 210. The fragrance 212 comprises a gel that will not run out of the tray 210. The fragrancer 220 of FIG. 9 is configured similarly to that of FIG. 4. However, the base includes a fan 222 that draws airflow 224 through openings in the simulated candle and over the fragrance 212 in the scent pack 200.

Any of the scent packs 22, 200 may alternatively include a simple annular base wall, for example a washer shaped surface, with no side walls and defining an opening to receive a portion of the fragrancer. The base wall may include a magnet as described above. The base wall may also be made of a magnetic material. For example, an annular washer shaped base wall may be formed from a magnetic material and a gel-type fragrance producing substance may be placed on the base wall.

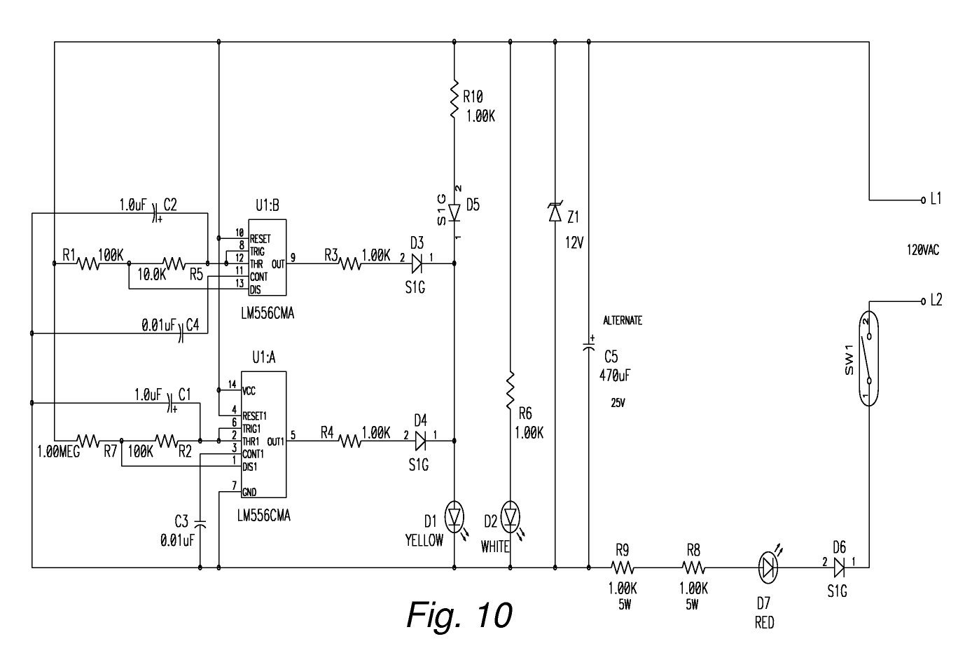

At its most rudimentary level, simulating a flame may be accomplished by changing the intensity of a lamp in a pseudo random pattern. For many applications this proves effective and provides a pleasing effect. FIG. 10 illustrates one possible implementation of this method. Two LED's are operated in parallel. A white LED is illuminated with a fixed current and hence a fixed luminous intensity. A second yellow LED is intensity modulated to simulate flame flicker. The two LED's are positioned in such a way as their light mixes to produce a color temperature similar to an actual flame.

To produce a varying intensity in this embodiment, two timers in the form of a stable multivibrator elements are run at slightly different frequencies and/or duty cycles and in such a way that their output circuitry modulate the current through the yellow LED. As they oscillate they add more or less current and change the intensity of the light emanating from the LED. Since they are asynchronous with one another, they will produce a pseudo random variation in this output.

In this embodiment power is provided by a 120VAC line input. The reed switch SW1 activates the circuitry when a magnet is placed in its proximity such as magnet 88 attached to the globe 24. A rectifier diode D6 produces a half-wave rectified signal through the LED D7, R8 and R9. D7 is intended to provide a level of circuit protection and is not used specifically for illumination. Capacitor C5 filters the half-wave rectified current producing a DC output voltage limited by the Zener diode Z1. This produces a regulated 12-Volt supply for the flame simulator circuitry.

The white LED D2 is powered through resistor R6 from the 12-Volt source, which produces a fixed current through D2. The yellow LED D1 is powered from three sources. R10 and D5 form a fixed current, which establishes a lower limit of intensity from LED D1. Two as table multivibrators, U1A and U1B, produce two slightly different frequency pulses which act through R4, D4 and R3, D3 respectively. D3 and D4 are “steering diodes” which prevent current from flowing back into U1A and U1B when these are in their low state.

There are four different states produced by the circuit of FIG. 10. The first state corresponds to both multivibrators, U1A and U1B, being in their low state and LED D1 being powered only through R10, D5. The second state corresponds to multivibrator U1A being in its high state and multivibrator U1B being in its low state such that LED D1 is powered through both R10, D5 and R4, D4. The third state corresponds to multivibrator U1B being in its high state and multivibrator U1A being in its low state such that LED D1 is powered through both R10, D5 and R3, D3. The fourth state corresponds to both multivibrators being in their high state such that LED D1 is powered through R10, D5; R3, D3; and R4, D4.

The resistance values shown in FIG. 10 for R3, R4, and R10 provide three different intensity values corresponding to 33%, 66%, and 100% of the maximum LED current. Since R3 and R4 are equal, both states two and three will produce 66% of the maximum LED current. When both outputs are at their low state, only the current through R10 will be supplied to the LED D1 and it will be illuminated at its lowest intensity. When one of the multivibrators transitions to a high output voltage level, the current through the LED doubles and the intensity increases roughly in proportion. When both multivibrator outputs are at a high level the current through the LED is three times that at its lowest level with a proportional increase in intensity. If the values are selected differently, then the intensity steps may be varied to produce a different range of intensity variations. If, for example, R3 and R10 are selected to be 1000 Ohms and R4 is selected to be 500 Ohms, then there would be four intensity levels set to 25%, 50%, 75% and 100% of full intensity depending on the combination of currents from the timer outputs. Other ratios may be used to vary the relative intensity variation for other effects such as an occasional higher intensity burst as happens when a more intense disturbance influences a candle flame.

The timing for the multivibrator U1A is controlled by resistors R2, R7 and capacitor C1. Likewise, resistors R1, R5 and capacitor C2 control the timing of the multivibrator UIB. Adjusting the value of these components will allow the timing to be varied and hence the flicker pattern duration.

This circuit is intended to be representative of one possible embodiment of this invention. Other configurations may be used without altering the spirit of the invention. For example, the circuit may be powered using a separate power supply, which may supply either AC or DC power. Additional timers may be added to produce further randomness and LED intensity levels.

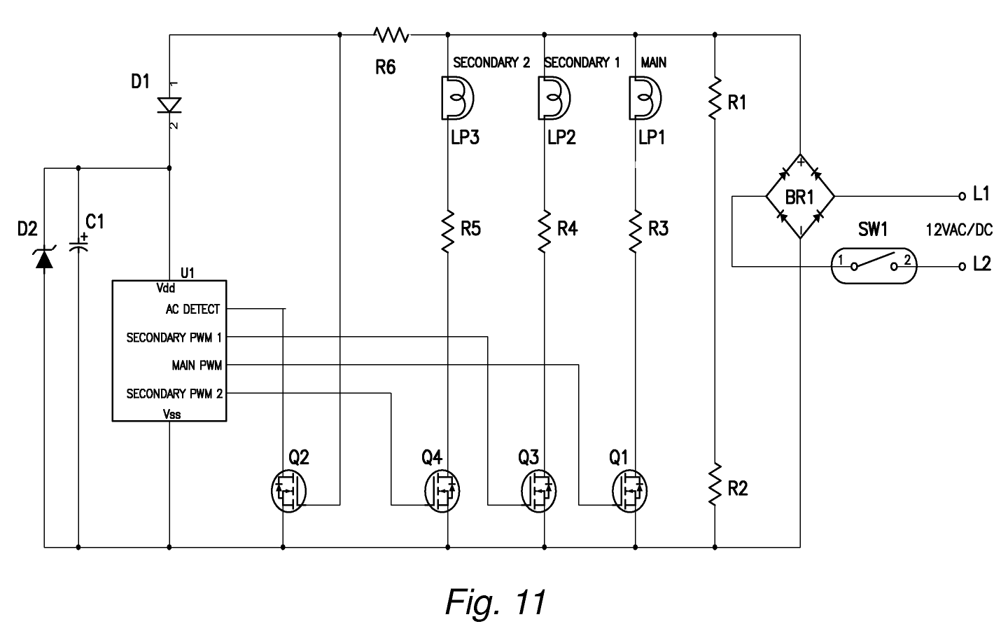

FIG. 11 illustrates an illustrative digital flame simulation circuit diagram. In this example, 12 volt AC or DC power is applied to inputs L1 and L2. Reed switch SW1 is controlled by the magnet 88 located in the globe 24. When switch SW1 is activated, current flows through the bridge rectifier BR1 to power the circuitry. The power resistors 46 are represented by R1 and R2. They act as the heating elements as previously described. Diodes D1 and D2, capacitor C1, and resistor R6 form the regulated low voltage power supply required by the microprocessor U1. The full wave rectified voltage produced by the bridge rectifier is detected by transistor Q2, which supplies a pulse to U1 at each zero crossing of the AC input. If the input power supply is DC, no pulse will be produced and the microprocessor U1 will determine that a DC supply is present. Lamp LP1 is the main lamp in the array of bulbs 34 of the flame simulation. Lamps LP2 and LP3 are secondary lamps in the array 34. Transistors Q1, Q3, and Q4 drive the lamps with a pulse width modulated (PWM) signal that allows the microprocessor to control the power dissipated in the lamps. Resistors R3, R4, and R5 limit the current in the bulbs.

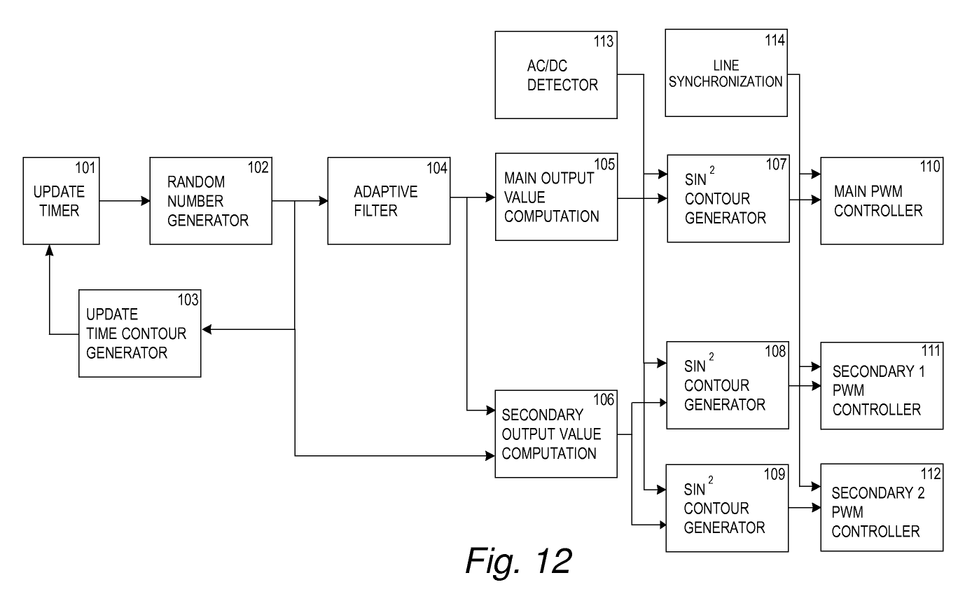

FIG. 12 shows a block diagram of the lamp control process operated by the microprocessor U1 of FIG. 11 to produce the candle flame simulation. The central controlling element of the process is the random number generator 102 which produces an 8-bit pseudo random number with uniform probability distribution. True random numbers would produce a series of numbers uniformly distributed over a defined interval with all numbers having equal probability of being generated. The generation of random numbers within a logic device such as a microprocessor necessarily limits the number sequence because the algorithms cannot be fully stochastic. For practical purposes then, generating a sequence of numbers which have equal probability of being produced but which may exhibit a pattern over a long time period is considered adequate when that repeating pattern is long enough to simulate a true random number. These long sequences are referred to as pseudo-random numbers since they do indeed have a pattern. There are numerous methods of performing this. One relatively simple method is described by Donald E. Knuth in Volume 2 of his series “The Art of Computer Programming”. In this reference he describes in detail a function which can produce pseudo random numbers. This function is defined by the following equation:

Xₙ₊₁=(aXₙ+c)mod m

Where Xₙ₊₁ is the next random number in a series, Xₙ is a current random number, a is a multiplier and c is an offset value. The function mod m is a division operation that produces the remainder value of the division by m. The constants a, c, and m may be chosen for the particular application. In this case a and c were chosen to be prime numbers and m was the byte length value of 256. When implemented this will produce a series of pseudo-random numbers in the range of 0 to 255 with each value having an equal probability of occurrence. The length of the sequence is sufficiently long that for this application it is effectively fully random.

The random number controls the simulated flicker rate and amplitude. The flicker rate is determined by the rate at which the random numbers are generated. More frequent changes will cause a faster flicker response. Likewise, less frequent changes will produce a slower flicker response. Since a candle flame is influenced by random turbulence, the simulated flame must likewise have a random flicker rate. Timer 101 is decremented at a fixed rate. When it reaches zero it triggers the generation of a new random number. This number is converted by the time contour generator 103 which produces a numeric value corresponding to the desired time interval to the next random number generation.

As has been described, a candle flame will attempt to achieve thermodynamic equilibrium and will exhibit a varying time response depending on the amplitude of a disturbance. A large disturbance will have a relatively short duration while a smaller disturbance will have a longer duration. The time contour generator 103 produces a relatively longer update time interval for values at the center of the random number range and will produce a shorter update time interval at either extreme. The random number generated is in the range of 0 to 255. Numbers near the center of this are defined to be nominal while numbers closer to either 0 or 255 will be more extreme. This method allows control of both the overall activity level of the simulated flame as well as the relative duration of the simulated disturbances.

The random number also controls the amplitude of the flicker. Since the random number will produce steps that are unpredictable and potentially large, a means of shaping the intensity transition from one number to the next must be incorporated. A real candle flame will vary smoothly in intensity with the disturbances. A filter is used to smooth the transitions from one intensity level to the next. Although a flame will vary smoothly, it will also respond to large dynamic changes differently than smaller changes. This is often observed as a brief but large flicker. To properly simulate this, the filter response must be adjusted when large transitions occur. Therefore, the filter response is implemented with adaptive filter 104. The basic filter is known to those skilled in the art as a single pole infinite impulse response (IIR) filter. The nominal filter cutoff frequency is set to approximately 0.8 Hz. This provides a smoothed transition from one brightness level to another, which more closely resembles the actual response of a candle. As the filter tends to average the random number input, it also tends to limit peak output. An actual candle flame will have brief instances where a peak value is reached followed by a more rapid decline toward the nominal output level. The filter is adjusted at certain high numeric values from the random number generator to respond more rapidly to these peak changes, and hence adapted based on the input value.

The filtered value is then used by the main output value computation 105 to produce a value suitable to the main PWM controller. We have found that the simulation produces a pleasing effect with the main output bulb set to have a moderate range of brightness variation from approximately 60% to 100% of full power output. This would correspond to approximately ⅓ of the numeric range of the filtered 8-bit random number value and an offset value of ⅔, providing the appropriate values for an 8-bit PWM output. A slightly smaller range was chosen for convenience so the computation performed by the main output value computation 105 scales the filtered value by ¼ so the duty cycle value varies from 75% to 100%. This is done by dividing the input value by 4 then adding a fixed offset of 191, which represent 75% of full scale. This variation was found to be suitable for use with either a single bulb or a multiple bulb implementation.

The secondary output value computation 106 also uses the filtered value but produces a somewhat more radical brightness range. In order to simulate the variation in flame height, the secondary bulbs are illuminated only when the input value exceeds some predetermined value. This threshold was determined to be approximately 33% of the full-scale intensity value. When the intensity exceeds this, the intensity value is scaled to optimally illuminate the secondary bulbs. When the bulbs are not illuminated, a low duty-cycle value is produced which keeps the filament at a temperature just below incandescence. Since incandescent lamps exhibit a non-linear positive temperature coefficient resistance, this keeps the resistance relatively high and limits the inrush current.

To simulate the lateral movement of the flame, one of the two secondary bulbs is selected for illumination based on one bit in the random number while the second bulb is left off. The bulbs alternate in a random pattern. This provides both a random lateral and vertical synthetic movement. This could also be implemented with additional filter elements that produce a smooth transition between the two secondary bulbs and thus an even more realistic simulation of lateral flame movement.

To vary the intensity of the bulbs, the PWM controllers 110, 111, and 112 generate a variable duty cycle waveform that is used to directly drive the bulbs. PWM digital-to-analog converters are known in the electronic arts to require minimum external circuitry to generate an analog signal from its digital representation within a microprocessor or other digital device. In the illustrative example, the bulbs are directly driven from the PWM where their light output is approximately proportional to the duty cycle.

It is desirable to reduce manufacturing costs of the flameless candle. One way this is achieved in the present invention is to eliminate as many components, especially relatively expensive components, as is feasible. One opportunity for reducing the component cost is in the power supply. Typically, lamps are more easily controlled when powered by DC. However, this would require a relatively large and expensive capacitor since the current drawn by the bulbs is relatively high. If the bulbs are driven by AC or rectified but unfiltered AC, this capacitor could be eliminated. However, this poses some difficulties.

With a DC power source, the intensity level varies approximately in direct proportion to the duty cycle of the PWM output. When AC power is used, the PWM output is no longer linearly proportional to the duty cycle due to the sinusoidal voltage being applied to the bulbs. Further, if the PWM is not synchronous with the AC power an interaction known as a beat frequency is produced which develops a highly undesirable effect. To avert this, the PWM output is first synchronized to the line frequency with a period equal to half that of the AC line. Then the PWM period is adjusted to produce equal steps of power delivered to the bulbs.

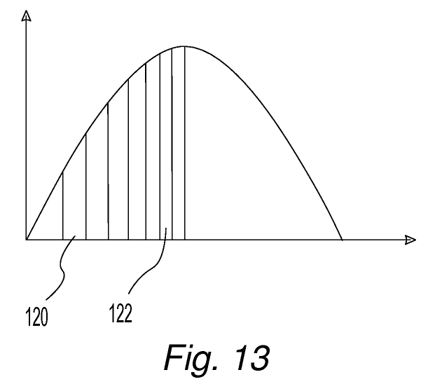

Since the power dissipation in the bulb is proportional to the square of the voltage applied, the power dissipation in the bulb is proportional to the square of the sine of the time during the input sine wave. Since the AC is full-wave rectified, only half of the sine wave cycle need be considered. The linear PWM value is converted to a value in which each increment in value is proportional to equal power levels. The power level increments correspond directly to equal areas under a sine curve. This is illustrated in FIG. 13 in which area 120 is the same as 122.

Selection of the AC or DC modes is done by a zero crossing detection system that senses when AC is applied. Transistor Q2 is connected to the rectified and unfiltered power source. When the voltage approaches a zero crossing it will drop below the threshold of Q2 and the transistor will turn off. This produces a pulse on the AC detect input to the microcontroller which is then used to synchronize the PWM. When operated on DC, Q2 will not change state which indicates to the microcontroller the presence of a DC power source.

Although examples of a flameless candle and its use have been described and illustrated in detail, it is to be understood that the same is intended by way of illustration and example only and is not to be taken by way of limitation. The invention has been illustrated configured with particular optional elements and circuit components. However, the flameless candle may be configured in a variety of ways and with varying circuit elements. For example, the flame simulation, trim ring, scent pack, globe, and globe insert, may be configured in any combination with one or more of the elements omitted. Likewise, analog or digital flame simulation circuits may be used. Also, the number of bulbs may be varied for different effects or cost targets. For example, a single bulb may be used in one embodiment to minimize cost. Two or more bulbs may be used in other instances to increase realism. Other kinds of light sources may be used in place of the illustrative incandescent bulbs. For example, the use of an LED light source may be advantageous in applications requiring batteries due to the lower power consumption of LED's versus incandescent bulbs.

The illustrative digital simulation circuit makes use of a small logic device and minimal external circuitry. All of the timing and lamp control is incorporated within the logic device. In the illustrative example, a microcontroller is used to generate and process the signals. Those skilled in the art will recognize that the same processes implemented by the microcontroller could also be implemented within a programmable logic device or in an application-specific integrated logic device. Such devices my also include the external components, such as transistors, without departing from the spirit of this invention.

Accordingly, variations in and modifications to the flameless candle and its use will be apparent to those of ordinary skill in the art, and such modifications and equivalents are encompassed in the invention.

What is claimed is:

1. A fragrancer for dispersing fragrance into the air, the fragrancer comprising:
a base;
a modular scent holder interchangeably mounted to the base, the scent holder having a base wall defining an opening able to receive a portion of the base, and
a volatile fragrance producing substance supported by the modular scent holder.

2. The fragrancer of claim 1, wherein the modular scent holder comprises an inner side wall extending away from the base wall, and an outer side wall extending away from the base wall, the base wall, inner side wall, and outer side wall defining an annular tray surrounding the opening able to contain the volatile fragrance producing substance.

3. The fragrancer of claim 2 wherein the base wall comprises an annular bottom wall between the inner and outer side walls, the inner and outer side walls extending upwardly from the bottom wall to define an upwardly facing annular cup, the inner side wall extending upwardly further than the outer side wall to define an upwardly projecting hollow extension communicating with the opening.

4. The fragrancer of claim 2 wherein the base wall comprises an annular top wall between the inner and outer side walls, the inner and outer side walls extending downwardly from the top wall to define a downwardly facing annular cup, the inner side wall extending upwardly above the top wall to define an upwardly projecting hollow extension communicating with the opening.

5. The fragrancer of claim 2 wherein the inner side wall defines a handle usable to manipulate the scent holder.

6. The fragrancer of claim 2 wherein the base includes a light producing element and the scent holder is receivable by the base with the light producing element extending through the opening in the base wall.

7. The fragrancer of claim 6 wherein the inner side wall forms a light diffuser operable to diffuse the light from the light producing element.

8. The fragrancer of claim 7 wherein the inner side wall is generally flame shaped.

9. The fragrancer of claim 6 wherein the inner side wall defines a light filter operable to change the color appearance of the at least one light producing element.

10. The fragrancer of claim 1 further comprising a power source and a powered device, the modular scent holder and base defining an interlock operable to enable power to the powered device when the modular scent holder is seated on the base and operable to interrupt power to the powered device when the modular scent holder is unseated from the base.

11. The fragrancer of claim 10 wherein the powered device comprises a heat source operable to heat the modular scent holder.

12. The fragrancer of claim 10 wherein the powered device comprises a light source.

13. The fragrancer of claim 10 wherein the powered device comprises a fan.

14. The fragrancer of claim 10 wherein the interlock comprises a switch type selected from the group consisting of pressure sensitive, magnetic, and photonic.

15. The fragrancer of claim 10 wherein the modular scent container includes a magnet mounted to the modular scent holder.

16. The fragrancer of claim 1 further comprising a modular globe interchangeably mounted to the base and surrounding the scent holder, the modular globe being provided in a variety of decorative appearances and being interchangeable by a consumer to vary the decorative appearance of the fragrancer.

17. The fragrancer of claim 1 further comprising a globe, a power source, and a powered device, the globe and base defining a switch operable to enable power to the powered device when the globe is in a first position and interrupt power to the powered device when the globe is in a second position.

18. The fragrancer of claim 17 wherein the powered device is a light source and the globe is mounted to the base for rotation relative to the base and surrounding the scent holder and light source, the light source being responsive to rotation of the globe on the base to change the light output from the light source.

19. The fragrancer of claim 1 further comprising a globe and a globe insert, the globe being mounted to the base and surrounding the scent container, the globe insert being interchangeably mounted to the globe and provided in a variety of decorative appearances and being interchangeable by a consumer to vary the decorative appearance of the fragrancer.

20. The fragrancer of claim 1 further comprising a modular trim ring interchangeably mountable to the base, the modular trim ring being provided in a variety of decorative appearances and being interchangeable by a consumer to vary the decorative appearance of the fragrancer.

21. The fragrancer of claim 1 further comprising a globe mounted to the base for rotation relative to the base and surrounding the scent holder, the fragrancer being responsive to rotation of the globe on the base to change its fragrance output.

22. A modular scent holder interchangeably mountable to a fragrancer, the modular scent holder comprising a first wall defining an opening able to receive a portion of the fragrancer and the first wall being able to support a volatile fragrance producing substance.

23. The modular scent holder of claim 22 further comprising:
a second wall extending away from the first wall; and
a third wall extending away from the first wall, the first, second, and third walls defining an annular tray surrounding the opening.

24. The modular scent holder of claim 24 further comprising a magnet mounted to the modular scent holder.

25. The modular scent holder of claim 24 wherein the second wall extends away from the first wall further than the third wall to define a hollow extension.

26. The modular scent holder of claim 25 wherein the extension is generally flame shaped.

27. A fragrancer for dispersing fragrance into the air, the fragrancer comprising:
a base;
a light producing element mounted to the base;
a heater;
a power source;
a modular scent container having a first wall and an opening through the first wall, a second wall extending away from the first wall, and a third wall extending away from the first wall, the first, second, and third walls defining an annular tray surrounding the opening, the scent container being interchangeably mountable to the base with the second wall surrounding the light producing element, the modular scent container and base defining an interlock operable to enable power to the heater when the modular scent container is seated on the base and operable to interrupt power to the heater when the modular scent container is unseated from the base.

28. A fragrancer for dispersing fragrance into the air, the fragrancer comprising:
a base;
a modular scent holder interchangeably mounted to the base;
a power source; and
a powered device, the modular scent holder and base defining an interlock operable to enable power to the powered device when the modular scent holder is seated on the base and operable to interrupt power to the powered device when the modular scent holder is unseated from the base.
